(19) United States
(12) Patent Application Publication
(10) Pub. No.: US 2022/0042100 A1
(43) Pub. Date: Feb. 10, 2022

(54) QUANTIFYING FOREIGN DNA IN LOW-VOLUME BLOOD SAMPLES USING SNP PROFILING

(71) Applicant: WILLIAM MARSH RICE UNIVERSITY, Houston, TX (US)

(72) Inventors: David Yu ZHANG, Houston, TX (US); Xi CHEN, Houston, TX (US); Omid VEISEH, Houston, TX (US); Peng DAI, Houston, TX (US); Kerou ZHANG, Houston, TX (US)

(21) Appl. No.: 17/311,102

(22) PCT Filed: Dec. 5, 2019
(86) PCT No.: PCT/US19/64670

§ 371 Date: Jun. 4, 2021

Related U.S. Application Data
(60) Provisional application No. 62/775,673, filed on Dec. 5, 2018.

(51) Int. Cl. C12Q 1/6883; C12Q 1/6827; C12Q 1/6806

(52) U.S. Cl. CPC C12Q 1/6883; C12Q 2600/156; C12Q 1/6806; C12Q 1/6827

(57) ABSTRACT

Provided herein are methods for quantifying foreign cell-free DNA (cfDNA) via SNP profiling of low-volume blood sample. The methods allow for monitoring the status of organ transplant rejection through analysis of small volumes of patient capillary blood samples collected non-invasively with fingersticks or other devices. The methods also allow for guiding the dosage of immunosuppressant and for preparing for a new organ transplant in case of imminent organ failure.

### REFERENCE TO RELATED APPLICATIONS

The present application claims the priority benefit of U.S. provisional application No. 62/775,673, filed Dec. 5, 2018, the entire contents of which is incorporated herein by reference.

### STATEMENT REGARDING FEDERALLY SPONSORED RESEARCH

This invention was made with government support under Grant No. R01 HG008752 awarded by the National Institutes of Health. The government has certain rights in the invention.

### BACKGROUND

### 1. Field

The present invention relates generally to the fields of molecular biology and genotype profiling. More particularly, it concerns methods for quantifying foreign DNA in low-volume blood samples using SNP profiling.

### 2. Description of Related Art

Organ recipients receive immunosuppressant to reduce the chance of rejection after receiving transplantation of non-self (allograft) organs. The standard diagnostic test for organ rejection is biopsy. Compared to traditional invasive biopsy, noninvasive tests are safer and allow more frequent monitoring of status of the transplant organ. However, the noninvasive biomarkers for early organ transplant rejection is limited. Creatinine in urine is the gold-standard for evaluating kidney rejections, but the level of creatinine increases only after major damage to the kidneys has occurred. Other biomarkers are being investigated for specific type of organ/tissue transplants, including mRNA for kidney (Suthanthiran et al., 2013), exosomes for pancreatic islet and kidney (Vallabhajosvula et al., 2017; Park et al., 2017). The donor-derived single nucleotide polymorphisms (SNPs) in cell-free DNA (cfDNA) may serve as a general noninvasive biomarker for organ transplant rejection. Though a SNP panel consisting of less than 267 SNPs is developed for monitoring immunosuppressive therapies in a transplant recipient (U.S. Pat. Appln. Publn. No. 2016/0145682), at least 1 mL plasma sample is required due to the need of cfDNA isolation from plasma. New methods of monitoring transplant recipients are needed.

### SUMMARY

As such, provided herein are methods to detect and monitor organ transplant rejection by profiling the single nucleotide polymorphisms (SNPs) from small volume finger-stick blood sample (less than 200 μL) of an organ transplant recipient. Also provided herein are methods for selectively amplifying cfDNA from total DNA, methods for using the fragmentation sites of cfDNA as molecular barcodes, and methods of profiling SNPs using specialized hybrid capture probe panels, and methods of quantifying the fraction of cfDNA that is donor-derived.

In one embodiment, provided herein are methods of selectively amplifying short DNA fragments in a DNA sample that comprises both long and short DNA fragments, the methods comprising: (a) ligating a universal adaptor oligonucleotide to each end of the long and short DNA fragments, thereby generating adaptor-modified long and short DNA fragments, (b) selectively amplifying the adaptor-modified short DNA fragments by performing PCR with an extension time of between about 1 second and about 15 seconds (such as, for example, 1, 2, 3, 4, 5, 6, 7, 8, 9, 10, 11, 12, 13, 14, or 15 seconds) and using oligonucleotide primers that hybridize to the universal adaptor, thereby generating amplified short DNA fragments, and (c) performing size selection to isolate the amplified short DNA fragments. Size selection may comprise gel electrophoresis purification or beads-based purification. Size selection may be performed using Ampure XP beads, gel purification, or electrophoresis.

In some aspects, the short DNA fragments have a length between about 50 nucleotides and 400 nucleotides, such as, for example, about 50-375 nucleotide, about 50-350 nucleotides, about 50-325 nucleotides, about 50-300 nucleotides, about 50-275 nucleotides, about 50-250 nucleotides, about 50-225 nucleotides, about 50-200 nucleotides, about 75-400 nucleotides, about 75-375 nucleotide, about 75-350 nucleotides, about 75-325 nucleotides, about 75-300 nucleotides, about 75-275 nucleotides, about 75-250 nucleotides, about 75-225 nucleotides, about 100-400 nucleotides, about 100-375 nucleotides, about 100-350 nucleotides, about 100-325 nucleotides, about 100-300 nucleotides, about 100-275 nucleotides, about 100-250 nucleotides, about 150-400 nucleotides, about 150-375 nucleotides, about 150-350 nucleotides, about 150-325 nucleotides, about 150-300 nucleotides, about 200-400 nucleotides, about 200-375 nucleotides, about 200-350 nucleotides, or any range derivable therein. In some aspects, the short DNA fragments may have an average size of about 50, 55, 60, 65, 70, 75, 80, 85, 90, 95, 100, 125, 150, 175, 200, 225, 250, 275, 300, 325, 350, 375, 400 nucleotides, or any value derivable therein.

In some aspects, the PCR in step (b) is performed with an annealing time of between about 1 second and about 30 seconds, such as, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 seconds. In some aspects, the DNA sample comprises cell-free DNA (cfDNA). In some aspects, the short DNA fragments comprise cfDNA. In some aspects, the DNA sample comprises DNA extracted from total blood. In some aspects, the DNA sample is extracted from a buccal swab or urine.

In some aspects, prior to step (a), the long and short DNA fragments are subjected to end-repair. In some aspects, prior to step (b), the adaptor-modified long and short DNA fragments are column purified. In some aspects, the universal adaptors comprise, from 5′ to 3′, a region that complementary to the oligonucleotide primers and a region that is not complementary to the oligonucleotide primers. In some aspects, the size selection of step (c) comprises gel purification. In some aspects, the methods further comprise (d) sequencing the amplified short DNA fragments.

In some aspects, the sequencing in step (d) is next-generation sequencing. In certain aspects, the next-generation sequencing is paired-end sequencing or single-read sequencing. In certain aspects, the methods further comprise (e) enriching the amplified short DNA fragment sequences by (1) aligning the sequences to a reference genome to determine the amplicon length and (2) removing any sequences with an amplicon length greater than 400 nucleotides.

In one embodiment, provided herein are methods of analyzing single nucleotide polymorphisms (SNPs) in a DNA sample, the method comprising: (a) hybridizing the DNA sample to a mixture of hybrid-capture probes, wherein at least 80%, at least 85%, at least 90%, at least 95%, or all of the hybrid-capture probes correspond, independently, to a genomic region having a SNP with a population minor allele frequency of greater than 25%, wherein each genomic region: (1) occurs no more than 10 times in the genome; (2) has a GC content of between about 0.25 and about 0.75; and (3) does not contain any string of a single base that is longer than 4 nucleotides, thereby generating capture probe-bound DNA; (b) isolating the hybrid-capture probe-bound DNA; (c) ligating a universal adaptor oligonucleotide to each end of the hybrid-capture probe-bound DNA; (d) amplifying the hybrid-capture probe-bound DNA using primers that hybridize to the adaptor sequences, thereby generating amplified DNA; and (e) sequencing the amplified DNA.

In some aspects, each genomic region comprises the 80 nucleotides surrounding the SNP. In some aspects, each genomic region within 40 nucleotides of the targeted SNP is unique in the genome or has a copy number of less than ten in the genome. Uniqueness and copy number may be evaluated using tools such as, for example, the Basic Local Alignment Search Tool (BLAST) from NCBI. In some aspects, the method analyzes between about 500 and about 1,000,000 SNPs, such as, for example, at least 500, 600, 700, 800, 900, 1,000, 1,500, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 600,000, 700,000, 800,000, or 900,000 and at most 600, 700, 800, 900, 1,000, 1,500, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000, or any range derivable therein. In some aspects, the hybrid-capture probes are biotinylated. In some aspects, the hybrid-capture probes are hybridized to a biotinylated oligonucleotide.

In some aspects, the DNA sample comprises cell-free DNA (cfDNA). In certain aspects, the cell-free DNA is isolated from whole blood. In certain aspects, the cfDNA is amplified prior to step (a). In some aspects, the DNA sample is amplified prior to step (a), thereby generating an amplified double-stranded DNA sample. In certain aspects, the DNA sample is amplified according to a method of any one of the present embodiments.

In some aspects, the short DNA fragments have a length between about 50 nucleotides and 400 nucleotides, such as, for example, about 50-375 nucleotide, about 50-350 nucleotides, about 50-325 nucleotides, about 50-300 nucleotides, about 50-275 nucleotides, about 50-250 nucleotides, about 50-225 nucleotides, about 50-200 nucleotides, about 75-400 nucleotides, about 75-375 nucleotide, about 75-350 nucleotides, about 75-325 nucleotides, about 75-300 nucleotides, about 75-275 nucleotides, about 75-250 nucleotides, about 75-225 nucleotides, about 100-400 nucleotides, about 100-375 nucleotides, about 100-350 nucleotides, about 100-325 nucleotides, about 100-300 nucleotides, about 100-275 nucleotides, about 100-250 nucleotides, about 150-400 nucleotides, about 150-375 nucleotides, about 150-350 nucleotides, about 150-325 nucleotides, about 150-300 nucleotides, about 200-400 nucleotides, about 200-375 nucleotides, about 200-350 nucleotides, or any range derivable therein. In some aspects, the short DNA fragments may have an average size of about 50, 55, 60, 65, 70, 75, 80, 85, 90, 95, 100, 125, 150, 175, 200, 225, 250, 275, 300, 325, 350, 375, 400 nucleotides, or any value derivable therein.

In certain aspects, the amplified double-stranded DNA sample is denatured prior to step (a), thereby generating an amplified single-stranded DNA sample. In certain aspects, the amplified double-stranded DNA sample is denatured by heating the amplified double-stranded DNA sample at a temperature of at least 80° C. (such as, for example, 80, 85, 90, 95, or 100° C.) for at least 2 minutes (such as, for example, 2, 3, 4, 5, 6, 7, 8, 9, 10, 11, 12, 13, 14, or 15 minutes). In certain aspects, the amplified double-stranded DNA sample is denatured by chemical denaturation. In certain aspects, the chemical denaturation comprises incubating the amplified double-stranded DNA sample with sodium hydroxide. In certain aspects, the amplified double-stranded DNA sample is denatured by enzymatic denaturation.

In some aspects, the sequencing in step (d) is next-generation sequencing. In certain aspects, the next-generation sequencing is paired-end sequencing. In certain aspects, the next-generation sequencing is single-read sequencing.

In some aspects, the isolating in step (b) comprises solid-phase capture of the hybrid-capture probe-bound DNA. In certain aspects, the solid-phase capture of the hybrid-capture probe-bound DNA comprises incubating the hybrid-capture probe-bound DNA with streptavidin-coated beads. In certain aspects, the isolating in step (b) further comprises separating, washing, and releasing the hybrid-capture probe-bound DNA. In certain aspects, separating comprises magnetic separation or centrifugation. In certain aspects, releasing comprises heating the captured hybrid-capture probe-bound DNA at least 80° C. (such as, for example, 80, 85, 90, 95, or 100° C.) for at least 2 minutes (such as, for example, 2, 3, 4, 5, 6, 7, 8, 9, 10, 11, 12, 13, 14, or 15 minutes). In certain aspects, the hybrid-capture probes further comprise an enzyme recognition moiety. In certain aspects, the enzyme recognition moiety is a cleavable base, such as, for example, deoxyuridine. In certain aspects, releasing comprises performing enzymatic cleavage of the enzyme recognition moiety. In certain aspects, releasing comprises incubating the captured hybrid-capture probe-bound DNA with a USER enzyme.

In one embodiment, provided herein are compositions comprising mixtures of hybrid-capture probes, wherein at least 80%, at least 85%, at least 90%, at least 95%, or all of the hybrid-capture probes correspond, independently, to a genomic region having a SNP with a population minor allele frequency of greater than 25%, wherein each genomic region: (1) occurs no more than 10 times in the genome; (2) has a GC content of between about 0.25 and about 0.75; and (3) does not contain any string of a single base that is longer than 4 nucleotides. In some aspects, each genomic region comprises the 80 nucleotides surrounding the SNP. In some aspects, each genomic region within 40 nucleotides of the targeted SNP is unique in the genome or has a copy number of less than ten in the genome. Uniqueness and copy number may be evaluated using tools such as, for example, the Basic Local Alignment Search Tool (BLAST) from NCBI. In some aspects, mixture comprises between about 500 and about 1,000,000 hybrid-capture probes, such as, for example, at least 500, 600, 700, 800, 900, 1,000, 1,500, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 600,000, 700,000, 800,000, or 900,000 and at most 600, 700, 800, 900, 1,000, 1,500, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000, or any range derivable therein. In some aspects, the hybrid-capture probes are biotinylated. In some aspects, the hybrid-capture probes are hybridized to a biotinylated oligonucleotide.

In one embodiment, provided herein are methods of determining the number of unique cfDNA fragments in a sample containing less than about 4 ng (such as, for example, less than about 1, 2, 3, 4, 5, 6, 7, 8, 9, or 10 ng) of cfDNA and/or correcting errors from amplification and sequencing, the method comprising: (a) amplifying the cfDNA fragments; (b) sequencing the amplified cfDNA fragments using paired-end next-generation sequencing; (c) aligning the sequences to a reference genome, and determining the start and end position of each sequenced cfDNA fragment; (d) separating the sequences by the genomic loci they are aligned to, and calling the fragment sequence based on majority vote of all the sequencing reads with the same start and end positions; and (e) counting the number of unique start and end positions from among the sequenced cfDNA fragments, thereby determining the number of cfDNA fragments at each genomic locus of interest corresponding to each different genotype in the sample. In some aspects, the start and end positions are determined by next-generation sequencing paired-end reads. The fragmentation sites may be represented by the first 2-50 nucleotides and the last 2-50 nucleotides in the cfDNA, the start and end coordinates relative to the reference genome, or the relative position of the start and end position relative to the SNP. The first 2-50 nucleotides of the cfDNA may be the first 2-50 nucleotides in the forward read, and the last 2-50 nucleotides of the cfDNA may be the first 2-50 nucleotides in the reverse read. In some aspects, the degenerate sequences are introduced by a ligation process and are used in combination with the fragmentation site as a unique molecular identifier.

In one embodiment, provided herein are methods of determining the number of unique cfDNA fragments in a sample containing more than 4 ng of cfDNA (such as, for example, more than about 1, 2, 3, 4, 5, 6, 7, 8, 9, or 10 ng) and/or correcting errors from amplification and sequencing, the method comprising: (a) ligating an adaptor nucleic acid to each end of each cfDNA fragment, wherein the adaptor nucleic acid comprises a degenerate sequence; (b) amplifying the adaptor-ligated cfDNA fragments; (c) sequencing the amplified cfDNA fragments using paired-end next-generation sequencing; (d) aligning the sequences to a reference genome, and determining the combined start and end position and degenerate sequence of each sequenced cfDNA fragment; (e) separating the sequences by the genomic loci they are aligned to, and calling the fragment sequence based on majority vote of all the sequencing reads with the same combined start and end positions and degenerate sequences; and (f) counting the number of unique combined start and end positions and degenerate sequences from among the sequenced cfDNA fragments, thereby determining the number of cfDNA fragments at each genomic locus of interest corresponding to each different genotype in the sample. In some aspects, the start and end positions are determined by next-generation sequencing paired-end reads. The fragmentation sites may be represented by the first 2-50 nucleotides and the last 2-50 nucleotides in the cfDNA, the start and end coordinates relative to the reference genome, or the relative position of the start and end position relative to the SNP. The first 2-50 nucleotides of the cfDNA may be the first 2-50 nucleotides in the forward read, and the last 2-50 nucleotides of the cfDNA may be the first 2-50 nucleotides in the reverse read.

In one embodiment, provided herein are methods of monitoring organ transplant rejection by SNP profiling, the method comprising: (a) extracting cell-free DNA and genomic DNA from a DNA sample obtained from an organ transplant recipient; (b) selectively amplifying short fragments of cell-free DNA using a methods of any one of the present embodiments; (c) obtaining sequence reads for at least 500 single nucleotide polymorphisms (SNPs) in the amplified cell-free DNA using a method of any one of the present embodiments; and (d) quantifying a fraction of the organ transplant donor-derived cell-free DNA versus the DNA of the organ recipient.

In some aspects, the cell-free DNA and genomic DNA are extracted from whole blood. In some aspects, the cell-free DNA and genomic DNA are extracted from a low-volume of the whole blood. The cell-free DNA and genomic DNA need not be, but may be, isolated from plasma. In some aspects, the extraction in step (a) further comprises plasma separation. In some aspects, the whole blood is venous blood. In some aspects, the whole blood is obtained from a finger-stick. In some aspects, the cell-free DNA and genomic DNA are extracted from a buccal swab. In some aspects, step (c) comprises simultaneously analyzing between 500 and about 1,000,000 SNPs, such as, for example, at least 500, 600, 700, 800, 900, 1,000, 1,500, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 600,000, 700,000, 800,000, or 900,000 and at most 600, 700, 800, 900, 1,000, 1,500, 2,000, 2,500, 3,000, 3,500, 4,000, 4,500, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000, or any range derivable therein.

In some aspects, step (d) comprises: (1) removing sequencing reads that comprise undetermined bases; and (2) determining the number of unique sequencing reads for each SNP. In certain aspects, determining the number of unique sequencing reads for each SNP comprises performing a method of any one of the present embodiments regarding using fragmentation sites as unique molecular identifiers. If the number of UMIs is smaller than a threshold that is set based on the input DNA amount, the UMI may be used for quantitation. If the number of UMIs is larger than the threshold, then the NGS read number may be used for quantitation.

If the donor genetic information is known, then the SNPs with identical genotype between the donor and the recipient may be discarded. Heterozygous SNPs in the recipient may also be discarded. If the donor genotype is unknown, then all the SNPs with “On-Recipient_ID %” larger than a threshold but smaller than another threshold may be used as distinguishing SNPs, wherein “Recipient_ID” is defined as the primary SNP genotype with the highest number of UMIs or NGS reads for a specific SNP locus. “On-Recipient_ID %” is defined as:

$$‘ On Recipient\_ID \% = \frac{Number of UMIs or Reads with ‘ Recipient\_ID ’}{Total number of UMIs or Reads at the SNP locus}$$

A cumulative donor score reflecting the donor-derived cfDNA fraction across all distinguishing SNPs may be calculated as follows:

$$Donor Score = \frac{\begin{matrix}Total number of UMIs or Reads \\ with SNP genotype other than ‘ Recipient\_ID ’\end{matrix}}{Total number of UMIs or Reads for all distinguishing SNPs}$$

In some aspects, the at least 500 SNPs consists of SNPs for which the organ transplant recipient is homozygous. In certain aspects, the at least 500 SNPs consists of SNPs for which the organ transplant recipient and the organ donor are not identical.

In some aspects, if the fraction of the short fragments of cell-free DNA that correspond to the DNA of the organ transplant donor is above a normal range or increases over time, then the organ transplant recipient is considered to be rejecting the transplanted organ.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Organ transplant rejection monitor by profiling SNPs from low-volume blood.

FIGS. 2A-B. The use of fragmentation sites of cfDNA from small-volume blood as unique molecular identifiers. FIG. 2A. The start and end coordinate of the cfDNA relative to the reference genome is different for each original cfDNA molecule, when the cfDNA molecule number is low. FIG. 2B. NGS reads with the same fragmentation sites are presumably derived from the same original molecule. The families of reads allow accurate quantitation for number of original molecules and removing reads with error from PCR amplification.

FIGS. 3A-C. FIG. 3A. Scheme of selective amplification of all the short DNA using universal primer from a mixture of DNA containing long DNA fragments. FIG. 3B. Agarose gel showing total DNA extracted from fingerstick capillary blood is mostly long genomic DNA. Fingerstick capillary blood is collected and the whole blood total DNA is extracted using QIAamp DNA Blood Mini Kit. The DNA is end repaired, dA-tailed and ligated with NEBNext adaptor and analyzed. FIG. 3C. Bioanalyzer trace showing cfDNA is amplified from total DNA, while long gDNA is not amplified during the PCR. The total DNA is extracted from 15 μL fingerstick capillary blood using QIAamp DNA Blood Mini Kit. The total DNA is end-repaired and ligated with NEBNext Adaptor for Illumina according to NEBNext UltraII protocol. The ligated product is amplified with Phusion polymerase and Illumina index primers i5 and i7.

FIG. 4. Design considerations of specialized hybrid capture probe panel for SNP profiling.

FIGS. 5A-D. Significance for uniqueness of the context genomic region around the targeted SNP. FIG. 5A. Proportion of the SNPs covered by NGS reads in the first panel, without BLAST checking. The SNPs are divided based on the copy number of the context sequence in human genome. About 20% of the probes in the first panel correspond to genomic regions the copy number of which are more than one in human genome. FIG. 5B. Poor NGS coverage uniformity for panel one. About 51% SNPs are not covered. FIG. 5C. Significantly improved coverage uniformity for panel two, in which the uniqueness for the context sequence of each SNP is checked by BLAST. FIG. 5D. Lorenz curves of SNP coverage analysis confirmed improved coverage uniformity of panel 2. Cumulative fraction of observed number of UMIs is plot against cumulative fraction of SNPs. The line 1 represents a hypothetically equal distribution across all the SNPs. The line 2 corresponds to the second SNP panel, and the line 3 corresponds to the first SNP panel. Line 3 deviates further from the perfect equality compared to line 2. The Gini Coefficients for lines 1, 2 and 3 are 0, 0.51 and 0.98 respectively.

FIGS. 6A-B. Number of SNPs needed for organ transplant rejection monitoring. FIG. 6A. 5556 SNPs need to be profiled to identify the presence of 0.1% donor-derived cfDNA in 50 μL finger-stick blood. FIG. 6B. The SNPs number is dependent on the input blood volume assuming a constant cfDNA concentration.

FIG. 7. An exemplary workflow of SNP profiling by specialized hybrid-capture probe panel. After end-repair, adaptor ligation and PCR amplification, the double-stranded DNA are mixed with the biotinylated specialized hybrid-capture probes and blockers. The mixture was incubated at 95° C. for 10 mins to denature double-strand DNA, followed by (65° C. 1 hr→47° C. 1 hr)×7, and 47° C. for 2 hr for hybridization. Streptavidin-coated magnetic beads are added to the mixture and incubated at 65° C. for 45 mins After beads washing to remove unbound DNA, the bound DNA molecules are released by a dual release mechanism involving USER enzyme treatment and 95° C. heat. Samples indices are added to the released DNA via PCR, and the products are sequenced by NGS.

FIG. 8. Workflow for quantifying donor-derived cfDNA fraction.

FIG. 9. Bioinformatics workflow to infer foreign molecule percentage. The genotype of donor is not required for quantitation. Only genotype of recipient is required. Normalization factor k is set to be 2 assuming the population VAF is around 0.5 for all the SNPs and assuming donor and recipient are not related at all.

FIG. 10. Inferred foreign molecule % is linear against the spike-in amount of sheared NA18562 into sheared NA18537.

FIG. 11. Boxplot of foreign molecule % in heathy people and non-rejection patients.

### DETAILED DESCRIPTION

Provided herein are methods of monitoring the status of organ transplant rejection by quantifying the fraction of donor-derived DNA via SNP profiling. These methods allow non-invasive organ transplant rejection monitoring from low-volume blood including finger-stick sample. These methods include the use of fragmentation sites of cfDNA from small-volume blood as unique molecular identifiers, selective amplification of short cfDNA using universal primers from a mixture of DNA containing genomic DNA, profiling between 500 and 1,000,000 targeted SNPs by NGS using a specialized hybrid capture probe panel, and an algorithm to quantify donor-derived cfDNA fraction.

### I. DEFINITIONS

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, 70, 75, 80, 85, 90, 95, or 100 nucleotides in length.

“Incorporating,” as used herein, means becoming part of a nucleic acid polymer.

The term “in the absence of exogenous manipulation” as used herein refers to there being modification of a nucleic acid molecule without changing the solution in which the nucleic acid molecule is being modified. In specific embodiments, it occurs in the absence of the hand of man or in the absence of a machine that changes solution conditions, which may also be referred to as buffer conditions. However, changes in temperature may occur during the modification.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” “Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.” In addition, the term “adaptor” can indicate a linear adaptor (either single stranded or double stranded) or a stem-loop adaptor. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

A “nucleic acid molecule” or “nucleic acid target molecule” refers to any single-stranded or double-stranded nucleic acid molecule including standard canonical bases, hypermodified bases, non-natural bases, or any combination of the bases thereof. For example and without limitation, the nucleic acid molecule contains the four canonical DNA bases—adenine, cytosine, guanine, and thymine, and/or the four canonical RNA bases—adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin. Examples of nucleic acid molecules include genomic DNA, cDNA, RNA, a DNA/RNA hybrid, amplified DNA, a pre-existing nucleic acid library, etc. A nucleic acid may be obtained from a human sample, such as blood, serum, plasma, cerebrospinal fluid, cheek scrapings, biopsy, semen, urine, feces, saliva, sweat, etc. A nucleic acid molecule may be subjected to various treatments, such as repair treatments and fragmenting treatments. Fragmenting treatments include mechanical, sonic, and hydrodynamic shearing. Repair treatments include nick repair via extension and/or ligation, polishing to create blunt ends, removal of damaged bases, such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

The term “non-complementary” refers to nucleic acid sequence that lacks the ability to form at least one Watson-Crick base pair through specific hydrogen bonds.

The term “blunt end” as used herein refers to the end of a dsDNA molecule having 5′ and 3′ ends, wherein the 5′ and 3′ ends terminate at the same nucleotide position. Thus, the blunt end comprises no 5′ or 3′ overhang.

“Cleavable base,” as used herein, refers to a nucleotide that is generally not found in a sequence of DNA. For most DNA samples, deoxyuridine is an example of a cleavable base. Although the triphosphate form of deoxyuridine, dUTP, is present in living organisms as a metabolic intermediate, it is rarely incorporated into DNA. When dUTP is incorporated into DNA, the resulting deoxyuridine is promptly removed in vivo by normal processes, e.g., processes involving the enzyme uracil-DNA glycosylase (UDG) (U.S. Pat. No. 4,873,192; Duncan, 1981; both references incorporated herein by reference in their entirety). Thus, deoxyuridine occurs rarely or never in natural DNA. Also contemplated are the nicking agents referred to as the USER™ Enzyme, which specifically nicks target molecules at deoxyuridine, and the USER™ Enzyme 2, which specifically nicks target molecules at both deoxyuridine and 8-oxo-guanine both leaving a 5′ phosphate at the nick location (see, U.S. Pat. No. 7,435,572). USER™ Enzyme is a mixture of uracil-DNA glycosylase (UDG) and the DNA glycosylase-lyase Endonuclease VIII. UDG catalyzes the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released. Non-limiting examples of other cleavable bases include deoxyinosine, bromodeoxyuridine, 7-methylguanine, 5,6-dihydro-5,6 dihydroxydeoxythymidine, 3-methyldeoxadenosine, etc. (see, Duncan, 1981). Other cleavable bases will be evident to those skilled in the art.

The term “degenerate” as used herein refers to a nucleotide or series of nucleotides wherein the identity can be selected from a variety of choices of nucleotides, as opposed to a defined sequence. In specific embodiments, there can be a choice from two or more different nucleotides. In further specific embodiments, the selection of a nucleotide at one particular position comprises selection from only purines, only pyrimidines, or from non-pairing purines and pyrimidines.

The term “ligase” as used herein refers to an enzyme that is capable of joining the 3′ hydroxyl terminus of one nucleic acid molecule to a 5′ phosphate terminus of a second nucleic acid molecule to form a single molecule. The ligase may be a DNA ligase or RNA ligase. Examples of DNA ligases include E. coli DNA ligase, T4 DNA ligase, and mammalian DNA ligases.

“Sample” means a material obtained or isolated from a fresh or preserved biological sample or synthetically-created source that contains nucleic acids of interest. Samples can include at least one cell, fetal cell, cell culture, tissue specimen, blood, serum, plasma, saliva, urine, tear, buccal swab, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals, other animals, plants, fungi, bacteria, and viruses.

As used herein in relation to a nucleotide sequence, “substantially known” refers to having sufficient sequence information in order to permit preparation of a nucleic acid molecule, including its amplification. This will typically be about 100%, although in some embodiments some portion of an adaptor sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

### II. NUCLEIC ACID ADAPTORS

In some embodiments, the present disclosure provides synthetic oligonucleotides that form double-stranded adaptors for use in the generation of nucleic acid libraries. The synthetic oligonucleotides that form the double-stranded adaptors can have a length of 20 to 100 nucleotides, particularly 50 to 80 nucleotides, such as between 60 and 70 nucleotides. Each double-stranded adaptor has a sense strand and an anti-sense strand. The 3′ end of the sense strand and the 5′ end of the anti-sense strand can form a blunt end or a staggered end. In particular aspects, the double-stranded regions have blunt ends.

The double-stranded nucleic acid adaptors further comprise at least one primer binding site with a known sequence. For example, the adaptor may comprise flow cell binding sequences, such as P5 and/or P7, or fragments thereof. Further, the adaptor can comprise part or all of sequencing primer sequences or their binding sites such as index sequencing primers for particular sequencing platforms (e.g., Illumina index primers).

### III. UNIQUE MOLECULAR IDENTIFIER (UMI) SEQUENCES

The term “unique molecular identifier” (or “UMI”) as used herein refers to a unique nucleotide sequence that is used to distinguish between a single cell or genome or a subpopulation of cells or genomes, and to distinguish duplicate sequences arising from amplification from those to which a UMI is linked to a target nucleic acid of interest by ligation prior to amplification, or during amplification (e.g., reverse transcription or PCR), and used to trace back the amplicon to the genome, cell, or nucleic acid fragment from which the target nucleic acid originated. A UMI can be added to a target nucleic acid by including the sequence in the adaptor to be ligated to the target. A UMI can also be added to a target nucleic acid of interest during amplification by carrying out reverse transcription with a primer that contains a region comprising the barcode sequence and a region that is complementary to the target nucleic acid such that the barcode sequence is incorporated into the final amplified target nucleic acid product (i.e., amplicon). A UMI can also be a feature present in the target nucleic acid itself, such as the fragmentation sites of a fragmented nucleic acid, e.g., a cell-free nucleic acid. The fragmentation sites can be identified by either the sequence at each end of the fragment or by the location of the end relative to a specific feature, such as a SNP, located within the fragment. The UMI may be any number of nucleotides of sufficient length to distinguish the UMI from other UMI. For example, a UMI may be anywhere from 4 to 20 nucleotides long, such as 5 to 11, or 12 to 20. The term “molecular identifier sequence,” “MIS,” “unique molecular identifier,” “UMI,” “molecular barcode,” “molecular tag sequence” and “barcode” are used interchangeably herein.

The present technology comprises the barcoding of nucleic acid molecules. Barcodes, also described as tags, indexing sequences, or identifier codes, include specific sequences that are incorporated into a nucleic acid molecule for identification purposes. For example, synthetic nucleic acid molecules can be joined with genomic DNA (gDNA) and/or cell-free DNA (cfDNA) by ligation and/or primer extension. Nucleic acid molecules may have multiple barcodes, such as, sequential or tandem barcodes. An example of a tandem barcode includes a first barcode coupled to at least one end of a DNA molecule by a ligation event (e.g., ligation to a synthetic adaptor) followed by a second barcode that is coupled to the DNA by primer extension (e.g., PCR), where the first barcode is proximal to the DNA molecule (closer to the insert) and the second barcode is distal to the DNA (further from the insert). Another example of a tandem barcode includes a first barcode that is the fragmentation site of a DNA molecule and a second barcode that is either coupled to the DNA by primer extension (e.g., PCR) by a ligation event (e.g., ligation to a synthetic adaptor). Methods of using adaptor ligation and primer extension, template extension, or PCR to add additional sequences are described, e.g., in U.S. Pat. No. 7,803,550, which is incorporated by reference herein in its entirety. These methods may be used in embodiments of the present invention to add a first and/or second barcode to a nucleic acid molecule.

Barcodes can be used to identify nucleic acid molecules, for example, where sequencing can reveal a certain barcode coupled to a nucleic acid molecule of interest. In some instances, a sequence-specific event can be used to identify a nucleic acid molecule, where at least a portion of the barcode is recognized in the sequence-specific event, e.g., at least a portion of the barcode can participate in a ligation or extension reaction. The barcode can therefore allow identification, selection or amplification of DNA molecules that are coupled thereto.

Fragments of genomic and/or cell-free DNA can be ligated to adaptors having a first set of barcodes, for example. The ligated adaptors and DNA fragments having the first set of barcodes can then be subjected to a primer extension reaction, template extension reaction, or PCR using a primer having a second set of barcodes. The resulting nucleic acid molecules each have one barcode from the first set of barcodes adjacent to one barcode from the second set of barcodes on at least one end of the nucleic acid molecule. The exact number of barcodes may be determined based on the particular application; for example, in some embodiments, the second barcode may use six bases to generate, e.g., 16 additional barcodes. Nonetheless, depending on the application and/or sequencing method 1, 2, 3, 4, 5, 6, 7, 8, 9, 10, 11, 12, 13, 14, 15, or 16 or more bases may be used to generate the second barcode. In some embodiments, at least 2, at least 3, or 3-16 bases can be used to generate a second barcode.

Barcoding is described, e.g., in U.S. Pat. No. 7,902,122 and U.S. Pat. Publn. 2009/0098555. Methods of using adaptor ligation and primer extension or PCR to add additional sequences are described, e.g., in U.S. Pat. No. 7,803,550, which is incorporated by reference herein in its entirety. Barcode incorporation by primer extension, for example via PCR, may be performed using methods described in U.S. Pat. No. 5,935,793 and U.S. Pat. Publn. 2010/0227329. In some embodiments, a barcode may be incorporated into a nucleic acid via using ligation, which can then be followed by amplification; for example, methods described in U.S. Pat. Nos. 5,858,656, 6,261,782, U.S. Pat. Publn. 2011/0319290, or U.S. Pat. Publn. 2012/0028814 may be used with the present invention. In some embodiments, one or more barcode may be used, e.g., as described in U.S. Pat. Publn. 2007/0020640, U.S. Pat. Publn. 2009/0068645, U.S. Pat. Publn. 2010/0273219, U.S. Pat. Publn. 2011/0015096, or U.S. Pat. Publn. 2011/0257031.

### IV. FURTHER PROCESSING OF TARGET NUCLEIC ACIDS

A. Repair of DNA Following Fragmentation

A nucleic acid molecule of interest can be a single nucleic acid molecule or a plurality of nucleic acid molecules. Also, a nucleic acid molecule of interest can be of biological or synthetic origin. Examples of nucleic acid molecules include genomic DNA, cDNA, cell-free DNA, RNA, amplified DNA, a pre-existing nucleic acid library, etc.

A nucleic acid molecule of interest may be subjected to various treatments, such as repair treatments and fragmenting treatments. Fragmenting treatments include mechanical, sonic, chemical, enzymatic, degradation over time, etc. Repair treatments include nick repair via extension and/or ligation, polishing to create blunt ends, removal of damaged bases such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

Preanalytical processing of nucleic acids for NGS requires fragmentation of the nucleic acid by mechanical or enzymatic shearing followed by ligation of adaptors specific to the analytical platform of choice. Some clinical samples, such as human plasma and serum, contain cell-free DNA that is already highly degraded. Whether fragmented artificially or naturally, there is significant damage to the ends of the nucleic acid (e.g., dsDNA), which must be repaired enzymatically to become competent for ligation. Ligation-competent nucleic acid ends are defined as intact blunt-ended double-stranded DNA ends that contain a phosphate at the 5′ terminus and a free hydroxyl group at the 3′ terminus.

Nucleic acids in a nucleic acid sample being analyzed (or processed) in accordance with the present invention can be from any nucleic acid source. As such, nucleic acids in a nucleic acid sample can be from virtually any nucleic acid source, including but not limited to genomic DNA, complementary DNA (cDNA), RNA (e.g., messenger RNA, ribosomal RNA, short interfering RNA, microRNA, etc.), plasmid DNA, mitochondrial DNA, etc. Furthermore, as any organism can be used as a source of nucleic acids to be processed in accordance with the present invention, no limitation in that regard is intended. Exemplary organisms include, but are not limited to, plants, animals (e.g., reptiles, mammals, insects, worms, fish, etc.), bacteria, fungi (e.g., yeast), viruses, etc. In certain embodiments, the nucleic acids in the nucleic acid sample are derived from a mammal, where in certain embodiments the mammal is a human. A nucleic acid molecule of interest can be a single nucleic acid molecule or a plurality of nucleic acid molecules. Also, a nucleic acid molecule of interest can be of biological or synthetic origin. Examples of nucleic acid molecules include genomic DNA, cDNA, cell-free DNA (cfDNA), RNA, amplified DNA, a pre-existing nucleic acid library, etc. In some aspects, the target nucleic acid is a double-stranded DNA molecule, such as, for example, human genomic DNA.

A nucleic acid molecule of interest may be subjected to various treatments, such as repair treatments and fragmenting treatments. Fragmenting treatments include mechanical, sonic, chemical, enzymatic, degradation over time, etc. Repair treatments include nick repair via extension and/or ligation, polishing to create blunt ends, removal of damaged bases such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

In the case of fragmented DNA (for example, cell-free DNA (cfDNA) from blood and/or urine) the reaction does not require a fragmentation. In particular, the isolated cfDNA may comprise fragments (e.g., of about 50 to 200 bp, particularly about 167 bp in length) and not need a fragmentation step prior to library preparation.

In some aspects, the plurality of nucleic acid molecules comprises nucleic acid fragments, such as gDNA subject to fragmentation. In some aspects, the shear force may be a hydrodynamic shear force, such as those generated by acoustic or mechanical means. Hydrodynamic shearing of a nucleic acid can occur by any method known in the art, including passing the nucleic acid through a narrow capillary or orifice, referred to as “point-sink” shearing (Oefner et al., 1996; Thorstenson et al., 1998: Quail, 2010), acoustic shearing, or sonication. The commercially available focused-ultrasonicators, in conjunction with miniTUBEs or microTUBEs (Covaris, Woburn, Mass.; U.S. Pat. Nos. 8,459,121; 8,353,619; 8,263,005; 7,981,368; 7,757,561), can randomly fragment DNA with distributions centered between 2-5 kb and 0.1-1.5 kb, respectively. Sonication subjects nucleic acid to hydrodynamic shearing forces (Grokhovsky, 2006; Sambrook et al., 2006). For example, the commercially available Bioruptor (Diagenode; Denville, N.J.; U.S. Patent Publn. No. 2012/0264228) use sonication to shear nucleic acids.

In certain aspects, a nucleic acid fragment, such as a short DNA fragment, may have a size of about 50 bp, about 100 bp, about 150 bp, about 200 bp, about 250 bp, about 300 bp, about 350 bp, about 400 bp, about 500 bp, about 1000 bp, or about 2000 bp. In certain aspects, the nucleic acid fragments, such as short DNA fragments, may have an average size of about 50 bp, about 100 bp, about 150 bp, about 200 bp, about 250 bp, about 300 bp, about 350 bp, about 400 bp, about 500 bp, about 1000 bp, or about 2000 bp. Nucleic acids may be, for example, RNA or DNA. Modified forms of RNA or DNA may also be used.

In certain embodiments, nucleic acid fragments that are processed according to aspects of the subject invention are to be pooled with nucleic acid fragments derived from a plurality of sources (e.g., a plurality of organisms, tissues, cells, or subjects), where by “plurality” is meant two or more.

An RNA molecule may be obtained from a sample, such as a sample comprising total cellular RNA, a transcriptome, or both; the sample may be obtained from one or more viruses; from one or more bacteria; or from a mixture of animal cells, bacteria, and/or viruses, for example. The sample may comprise mRNA, such as mRNA that is obtained by affinity capture.

Obtaining nucleic acid molecules may comprise generation of the cDNA molecule by reverse transcribing the mRNA molecule with a reverse transcriptase, such as, for example Tth DNA polymerase, HIV Reverse Transcriptase, AMV Reverse Transcriptase, MMLV Reverse Transcriptase, or a mixture thereof.

There are two main types of DNA end damage that result in DNA ends that are not competent for ligation: ends that are not blunt; and ends that lack a phosphate at a 5′-end and/or have a phosphate at a 3′-end.

The first type of damage can be repaired by the concerted action of a DNA polymerase that extends recessed ends in the presence of deoxynucleotide triphosphates (dNTPs) or a 3′ exonuclease that trims protruding 3′ ends to produce blunt ends. The most commonly used enzyme for this type of repair is T4Pol, which has both DNA polymerase and DNA 3′ exonuclease activities residing on the same protein. However, use of T4Pol may result in over-trimming, thus producing one or two base recessed ends that are not competent for ligation. Klenow has the same enzymatic activities as T4Pol but much weaker 3′ exonuclease than its counterpart. This property makes it a useful supplement to T4Pol for reducing the risk of over-trimming and making the blunt-end reaction more efficient.

The second type of damage can be repaired by enzymatic activities that transfer phosphates to the 5′ termini of DNA and remove phosphates from the 3′ termini of DNA, such as 3′ phosphatases and/or 3′ exonucleases that are not inhibited by the presence of 3′ phosphate, such as, for example, PNK. PNK transfers phosphate from deoxynucleotide triphosphates to the 5′ termini of DNA in a reversible reaction that depends on the concentration of dNTPs, i.e., high dNTP concentrations shift the equilibrium toward transfer to DNA while high concentrations of diphosphates stimulate the reverse reaction. PNK also has an intrinsic 3′-phosphatase activity that removes phosphate from the 3′ termini of DNA but this activity is often insufficient to achieve complete repair.

Those skilled in the art will realize that in the case that the target nucleic acid lacks a 3′-OH and/or has a naturally blocked, non-extendable 3′ terminus (such as, for example, a 3′ terminal phosphate, a 2′,3′-cyclic phosphate, a 2′-O-methyl group, a base modification, a backbone sugar or phosphate modification, etc.), the blocked 3′ terminus can be repaired or cleaved to expose a 3′-OH by enzymatic treatment to remove the blocking group prior to proceeding with the methods. In some aspects, repair of the 3′ ends of a target nucleic acid molecule may be performed by a polymerase (e.g., T4 DNA polymerase, Klenow fragment), a kinase (e.g., T4 polynucleotide kinase), a phosphatase (e.g., alkaline calf intestinal phosphatase), a 3′ exonuclease (e.g., exonuclease I, exonuclease III), and/or a restriction endonuclease. In this method, input DNA may be simultaneously fragmented, repaired, and ligated to adaptors. This is accomplished by incubating the input DNA with a polymerase (e.g., T4 DNA polymerase, Klenow fragment), a kinase (e.g., T4 polynucleotide kinase), a phosphatase (e.g., alkaline calf intestinal phosphatase), a 3′ exonuclease (e.g., exonuclease I, exonuclease III), a DNA ligase, and ligation adaptors. In other aspects, these reactions can also be performed sequentially such that the fragments under repair and then repaired fragments are incubated with a DNA ligase and ligation adaptors.

B. Amplification of DNA

A number of template-dependent processes are available to amplify the nucleic acids present in a given template sample. One of the best known amplification methods is the polymerase chain reaction (referred to as PCR™) which is described in detail in U.S. Pat. Nos. 4,683,195, 4,683,202, and 4,800,159 and in Innis et al., 1990, each of which is incorporated herein by reference in their entirety. Briefly, two synthetic oligonucleotide primers, which are complementary to two regions of the template DNA (one for each strand) to be amplified, are added to the template DNA (that need not be pure), in the presence of excess deoxynucleotides (dNTP's) and a thermostable polymerase, such as, for example, Taq (Thermus aquaticus) DNA polymerase. In a series (typically 30-35) of temperature cycles, the target DNA is repeatedly denatured (around 90° C.), annealed to the primers (typically at 50-60° C.) and a daughter strand extended from the primers (72° C.). As the daughter strands are created they act as templates in subsequent cycles. Thus, the template region between the two primers is amplified exponentially, rather than linearly.

C. Sequencing of DNA

Methods are also provided for the sequencing of the library of adaptor-linked fragments. Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing.

The nucleic acid library may be generated with an approach compatible with Illumina sequencing such as a Nextera™ DNA sample prep kit, and additional approaches for generating Illumina next-generation sequencing library preparation are described, e.g., in Oyola et al. (2012). In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.). Additional methods for next-generation sequencing methods, including various methods for library construction that may be used with embodiments of the present invention are described, e.g., in Pareek (2011) and Thudi (2012).

In particular aspects, the sequencing technologies used in the methods of the present disclosure include the HiSeg™ system (e.g., HiSeg™ 2000 and HiSeg™ 1000), the NextSeq™ 500, and the MiSeq™ system from Illumina, Inc. The HiSeg™ system is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. The MiSeq™ system uses TruSeq™, Illumina's reversible terminator-based sequencing-by-synthesis.

Another example of a DNA sequencing technique that can be used in the methods of the present disclosure is 454 sequencing (Roche) (Margulies et al., 2005). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection—no scanning, no cameras, no light—each nucleotide incorporation is recorded in seconds.

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

A further sequencing platform includes the CGA Platform (Complete Genomics). The CGA technology is based on preparation of circular DNA libraries and rolling circle amplification (RCA) to generate DNA nanoballs that are arrayed on a solid support (Drmanac et al. 2009). Complete genomics' CGA Platform uses a novel strategy called combinatorial probe anchor ligation (cPAL) for sequencing. The process begins by hybridization between an anchor molecule and one of the unique adaptors. Four degenerate 9-mer oligonucleotides are labeled with specific fluorophores that correspond to a specific nucleotide (A, C, G, or T) in the first position of the probe. Sequence determination occurs in a reaction where the correct matching probe is hybridized to a template and ligated to the anchor using T4 DNA ligase. After imaging of the ligated products, the ligated anchor-probe molecules are denatured. The process of hybridization, ligation, imaging, and denaturing is repeated five times using new sets of fluorescently labeled 9-mer probes that contain known bases at the n+1, n+2, n+3, and n+4 positions.

### V. KITS

The technology herein includes kits for analyzing single nucleotide polymorphisms (SNPs) in a DNA sample, for selectively amplifying short DNA fragments from a DNA sample that contains both short and long DNA fragments, and kits for monitoring organ transplant rejection by SNP profiling. A “kit” refers to a combination of physical elements. For example, a kit may include, for example, one or more components such as double-stranded nucleic acid adaptors, hybrid-capture probes, specific primers, enzymes, reaction buffers, an instruction sheet, and other elements useful to practice the technology described herein. These physical elements can be arranged in any way suitable for carrying out the invention.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow molded plastic containers into which the desired vials are retained. A kit will also include instructions for employing the kit components as well the use of any other reagent not included in the kit. Instructions may include variations that can be implemented.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—SNPs as Biomarker of Organ Transplant Rejection

Cell-free DNA (cfDNA) in the circulating blood plasma are typically derived from cells that died within the previous 30 minutes. cfDNA is continually excreted via urine, so it provides an accurate and up-to-date “snapshot” of the patient and the donated organ. When the organ from the donor is rejected and attacked by the immune system, the concentration of cfDNA derived from the dying rejected organ's cells will significantly increase. Since SNP differences are present between donor and recipient patient genome, the percentage of donor DNA can be inferred by profiling SNPs in cfDNA, which can be used to detect and quantify organ rejection in even early stages (FIG. 1).

### Example 2—Natural Unique Molecular Identifiers (UMI) of cfDNA from Low-Volume Blood

Finger-stick blood is convenient to collect, non-invasive, and patient-friendly. Because the cfDNA molecule number is very low in small-volume finger-stick blood, the intrinsic fragmentation site information of cfDNA can serve as unique molecular identifier (UMI). UMIs are a way to reduce the quantitation bias and polymerase error introduced during DNA amplification. This usually requires attaching a unique DNA barcode (UMI) to each original molecule before amplification. All NGS reads with the same UMI are presumably derived from the same original molecule.

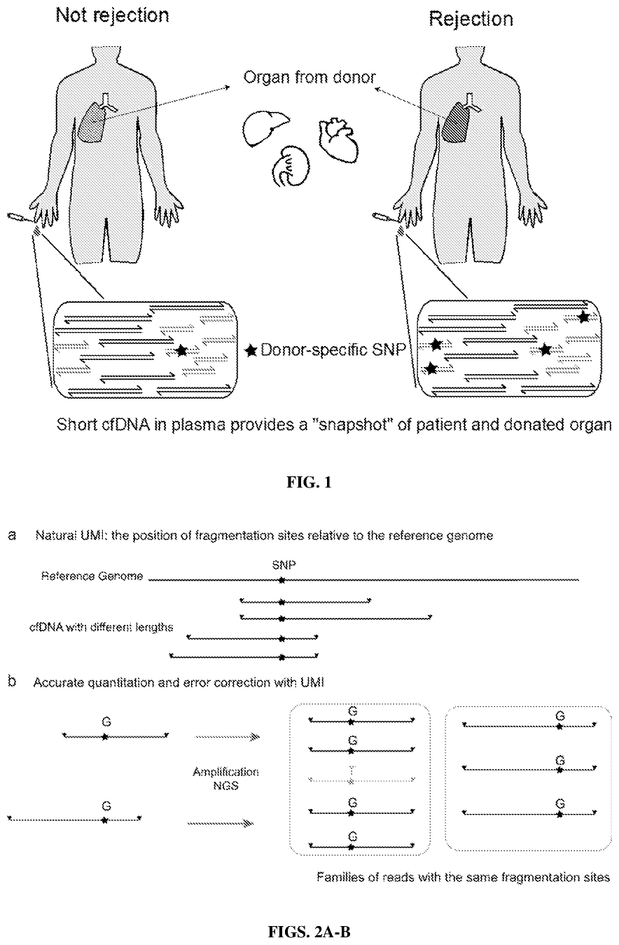

Fragmentation sites of cfDNA can be treated as unique molecular identifiers (FIG. 2). The number of possible combinations for start and end coordinates of the cfDNA relative to the reference genome is orders of magnitude larger than the cfDNA molecule number in 50 μL finger-stick blood. The average length of cfDNA is around 160 nucleotides. If all the DNA molecules covering a specific SNP site have a length of 160 nucleotides, then there are 160 different possible fragmentation sites. The number of possible fragmentation site combinations for cfDNA covering a specific SNP site should be at least 2,000 considering the cfDNA size distribution. If the cfDNA concentration in plasma is 2.5 ng/mL, the cfDNA haploid copy number is 15 in 50 μL blood. In this case, each molecule among the 15 will have distinct fragmentation sites, as indicated by a numerical simulation. The number of cfDNA will be elevated in the case of organ transplant rejection. In extreme cases, the molecule number may increase 10-fold from 15 to 150. But more than 95% of the original molecules still have distinct fragmentation sites. If the cfDNA haploid copy number is too high to be uniquely represented by fragmentation sites, such as when the molecule number is >1000, the NGS data will be processed without considering UMI.

The fragmentation site UMIs can be expressed in more than one way. The UMI can be shown as the start and end coordinates, such as (12300, 12460). The relative position of the start and end position relative to the SNP site is another way of labeling each molecule, such as (−120, +39). In addition, the first 2-50 nucleotide sequence and the last 2-50 nucleotide sequence of the cfDNA can be used.

### Example 3—Selective Amplification of all the Short DNA Using Universal Primers from a Mixture of DNA Containing Long DNA Fragments

It is essential to selectively amplify short cfDNA from total DNA when using finger-stick blood as the sample for organ transplant monitoring. Because of the presence of genomic DNA from leukocytes, DNA extracted from total blood is mostly genomic DNA with around 0.01% cfDNA. The usual cfDNA extraction requires separating plasma from buffy coat and erythrocytes in the total blood. If the blood sample volume is very low, such as for 20-50 μL finger-stick blood as an example, the cfDNA extraction process is not convenient and will result in significant loss. In addition, the plasma separation step is time-sensitive upon collecting the specimen (typically within one hour) and requires professional equipment and personnel. Selective amplification of cfDNA from the DNA extracted from total blood will circumvent the limitations resulting from cfDNA extraction.

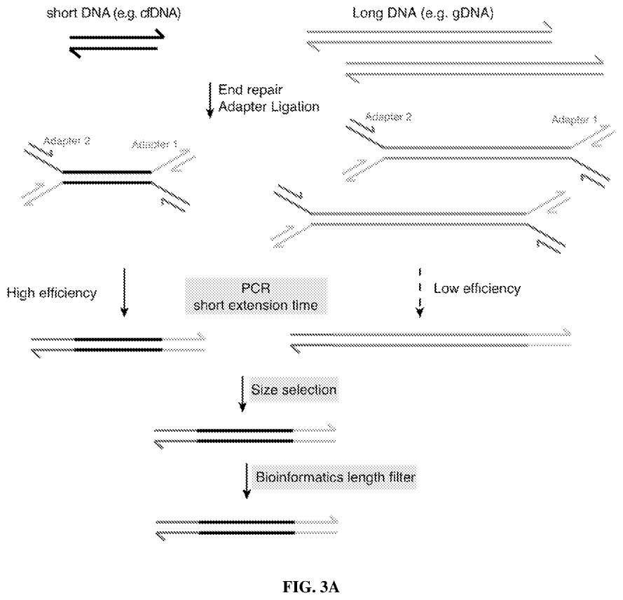

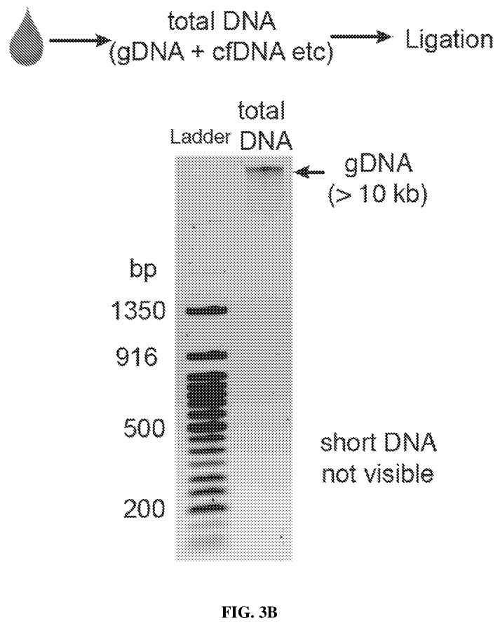

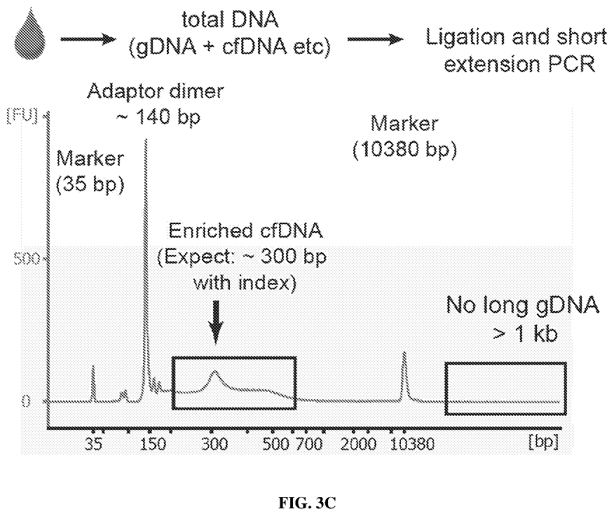

Short PCR extension time, size selection, and bioinformatics length filters are combined to selectively enrich short DNA (FIG. 3A). As an example to illustrate the enrichment process, 1 ng or 0.1 ng fragmented genomic DNA NA18537 with an average length of 100 bp was mixed with intact genomic DNA NA18562 in a 1:10,000 ratio as input. End-prep and adaptor ligation followed the protocol of NEBNext® Ultra™ II DNA Library Prep kit. After end-prep, universal adaptor ligation, and column purification, the ligated DNA was PCR amplified under short extension time. Prior to amplification, the ligated total DNA was analyzed by gel electrophoresis, which revealed that very little short DNA was present (FIG. 3B). The extension time for Phusion High-Fidelity DNA Polymerase is recommended to be 15-30 seconds per kb of amplicon. To selectively amplify DNA shorter than 1 kb, annealing time was set to be 10 seconds so that all of the short DNA is amplified exponentially, while long DNA is less efficiently amplified. Size selection was applied to the PCR product to remove DNA longer than 1 kb while maintaining the DNA shorter than 500 bp. The SNP information of the amplified DNA was profiled by a specialized hybrid capture probe panel, the design considerations of which are described in Example 4. Because human genomic DNA is mostly longer than 10 kb, the short-fragmented DNA or cfDNA is significantly enriched during PCR and size selection. As summarized in Table 1, the fraction of molecules from NA18537 is more than 10% under both of the two sample inputs, as indicated by the selected 53 SNP sites with different genotypes for NA18537 and NA18562. A more than 1000-fold enrichment of short sheared NA18537 was observed. By aligning to the reference, the length of the original molecule can be inferred from paired-end NGS reads. The data could be further processed to improve the enrichment performance via removing NGS reads corresponding to long fragments.

To show that these methods can enrich cfDNA from total DNA, an enrichment study was performed (FIG. 3C). The ligated total DNA from 15 μL fingerstick capillary whole blood were amplified using the described methods following the ligation-amplification protocol and characterized by High sensitivity DNA Bioanalyzer. The annealing time was 20 seconds and the extension time was 20 seconds. Because Illumina index primers i5 and i7 were used for amplification, the expected length for cfDNA after ligation and amplification was about 300 bp. A peak at 300 bp was clearly observed, with fewer amplicons with lengths of 350-600 bp. A flat baseline was observed for long genomic DNA length, confirming the removal of long gDNA. The amplicons with lengths between 350-600 bp might be derived from tiny amounts of short genomic DNA fragments either naturally existing in the cells or introduced during the experiment.

### Example 4—Design Considerations of Specialized Hybrid Capture Probe Panel for SNP Profiling

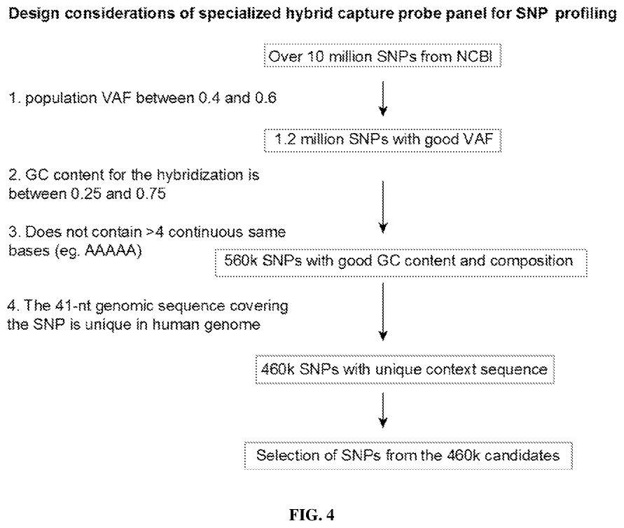

The SNP panel is designed to enable distinguishing different human genomes based on SNP signature. Each probe in the panel must be highly specific to the desired SNP loci in the human genome. The SNP panel selection scheme is summarized in FIG. 4.

First, SNPs are chosen based on the population variant allele frequencies. SNPs are natural variations in the genome. The 1000 Genomes project provides information including population variant allele frequency on over 10 million different SNP sites. About 1.2 million SNP sites have variant allele frequency (VAF) between 0.4 and 0.6, and about 3.2 million of them have VAF between 0.25 and 0.75. The probability of two unrelated individuals matching perfectly at a SNP locus with 40% variant population frequency is roughly (0.4*0.4)²+(0.4*0.6*2)²+(0.6*0.6)²=38.6%, so that the SNP has a 61.4% chance to distinguish the two individuals. Because a small allele ratio change from donor-derived DNA may be difficult to be confidently called at a heterozygous SNP in the recipient, the probability of the case in which the recipient is homozygous and the donor is different from the recipient is considered. At a SNP locus with 40% or 60% population VAF, the stringent distinguishing probability is roughly 0.4²*(1-0.4²)+0.6²*(1-0.6²)=36.5%. The probability is enhanced slightly to (0.5²*(1-0.5²))*2=37.5% at a SNP locus with 50% population VAF.

The detailed information (chromosome number, SNP position, reference sequence, alternative sequence, allele frequency, and reference genome) of all the 1.2 million SNP loci with the allele frequency between 0.4 and 0.6 in the whole human genome was obtained from the NCBI SNP database. Then the 80-nt context sequence (the 40 nucleotides before and 39 nucleotides after the single-nucleotide SNP position) was downloaded from NCBI Genome Reference Consortium Human Build 37 (GRCh37, hg19) as the hybridization domain candidates for further selection.

Second, the SNP probe panel is chosen based on GC content and sequence composition. The GC content for the 80-nt hybridization domain must be between 0.25 and 0.75. The hybridization domain should not contain 5 or more than 5 continuous same bases for fidelity considerations of probe synthesis. Around 560,000 SNPs satisfy the requirements.

Third, the SNPs are further filtered based on the uniqueness of the genomic region around the targeted SNP. For specificity considerations, the 41-nt genomic context sequence covering the SNP, including the 20-nt before and 20-nt sequence after the SNP, is evaluated by Basic Local Alignment Search Tool (BLAST) from NCBI to avoid any genomic regions with a copy number>10 in human genome. Around 460,000 SNPs have unique context sequence (copy number=1) in the genome.

The final SNP panel is selected from the 460,000 SNPs that meet all the requirements. To minimize the likelihood of genetic linkage, the SNPs are broadly spaced across the 22 pairs of human autosomes. Each of the SNPs in the panel are at least 200-nt away from each other.

### Example 5—Significance for Checking Uniqueness for Context Sequence of the Targeted SNP

The uniqueness of the genomic region around the targeted SNP is required for a successful specialized hybrid capture probe panel. To evaluate the significance of uniqueness, two SNP panels are compared in a hybrid capture NGS experiment. 1 ng fragmented NA18537 genomic DNA, which corresponds to about 300 haploid genomic copies, is used as sample input.

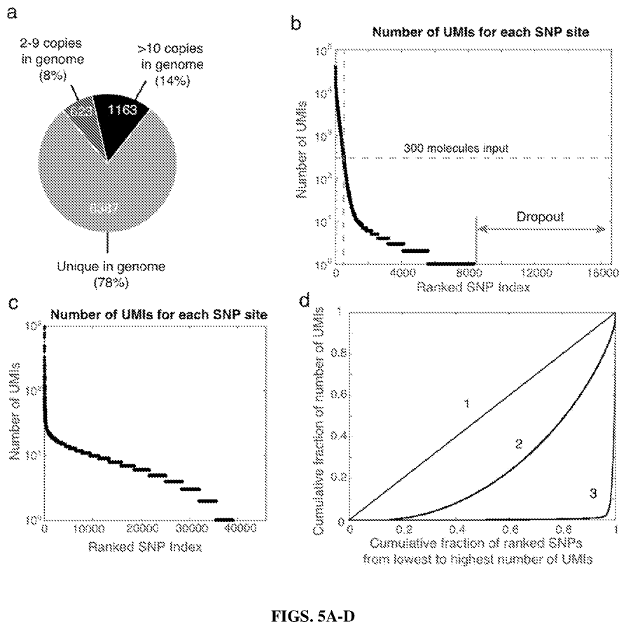

The first probe panel satisfied all the design considerations except that the uniqueness of the context sequence around the SNP is not considered. The panel consisted of 12,000 probes covering 16,632 SNPs. For the first panel, the SNPs covered by NGS reads are grouped as three classes based on the uniqueness of its 41-nt context sequence covering the SNP (FIG. 5A). Only 6387 (78%) of the SNPs are within unique context genomic sequence. However, the copy number of the SNP context sequence for 623 (8%) SNP loci is 2-9, while the copy number of the SNP context sequence is >10 for 1163 (14%) SNP loci.

Non-specific probes result in poor NGS reads coverage uniformity and potential artifact SNP genotype. Coverage uniformity is the distribution of on-target NGS reads that correspond to different SNP loci. Because the 22% non-specific probes consume more than 99% of the NGS reads, only 8,173 out of the 16,632 SNPs are covered from about 3 million NGS reads and the rest are dropout. The observed number of original molecules, considering fragmentation sites as UMI, are significantly different between unique probes and non-specific probes (FIG. 5B). The original molecule number for each SNP within a unique genomic region is between 1 and 138. However, the molecule number per each SNP within non-specific genomic region is 1,202 on average, which is more than the estimated input molecule number (300). The 514 SNP loci corresponding to more than 300 molecules are all within non-specific genomic regions. Non-specific sequences interfere with the SNP calling for desired loci and could result in artifact SNP genotype.

The second SNP panel consisted of 45,842 SNPs in which the uniqueness for context sequence of each targeted SNP was ensured by BLAST, resulting in a significantly improved coverage uniformity (FIG. 5C). 38,941 out of 45,842 SNPs were covered by about 4 million NGS reads; only 15% of the SNPs are dropout. Lorenz curves of SNP coverage analysis further confirmed the improvement of coverage uniformity of the second SNP panel. Cumulative fraction of observed number of UMIs against cumulative fraction of SNPs is shown for the two panels (FIG. 5D). The straight line (line 1) represents a hypothetically equal distribution across all the SNPs, line 2 corresponds to the second SNP panel, and the line 3 corresponds to the first SNP panel. Line 3 significantly deviates further from the perfect equality compared to line 2. The Gini Coefficients for lines 1, 2, and 3 are 0, 0.51, and 0.98, respectively, confirming that the SNP panel without considering context sequence uniqueness leads to deteriorated coverage uniformity.

### Example 6—Number of SNPs Needed for Organ Transplant Rejection Monitoring

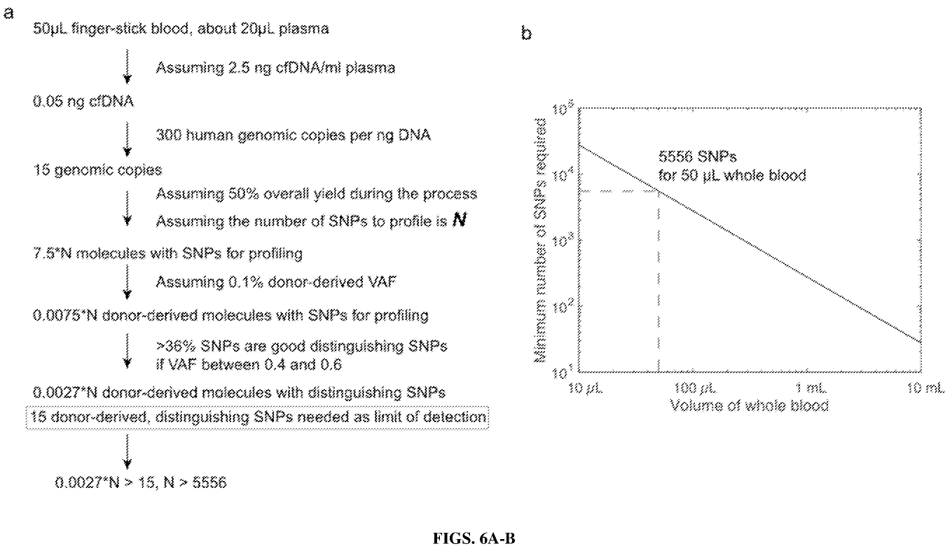

Thousands of SNPs need to be profiled to identify donor-derived cfDNA fraction from small volume finger-stick blood. As illustrated in FIG. 6A, assuming the finger-stick whole blood volume is 50 μL, the cfDNA concentration is 2.5 ng/mL in plasma, and assuming a 50% overall yield during the process of DNA extraction and amplification, 7.5 haploid genomic copies will be extracted. The number of molecules to be profiled is 7.5*N, wherein N is the number of SNPs in the specialized panel. Because all the SNPs in the panel have a population VAF between 0.4 and 0.6, >36% of the SNPs will be good distinguishing biomarkers for any of two unrelated people. Assuming donor-derived DNA VAF is 0.1%, the number of donor-derived molecules with distinguishing SNPs will be:

7.5*N*0.1%*36%=0.0027N

The limit of detection (LOD) was set to be 15 donor-derived distinguishing SNPs, so that 0.0027*N should be >15; the number of SNPs is larger than 5,556.

Because the DNA molecule number is proportional to the volume of blood, the number of SNPs needed for monitoring organ transplant rejection is dependent on the blood sample volume (FIG. 6B).

### Example 7—SNP Profiling by Hybrid Capture

Amplification of biotinylated specialized hybrid-capture probes for SNP profiling. A non-modified single stranded DNA pool containing 80-nt hybridization domains and two 30-nt universal domains for amplification is ordered from Twist Bioscience. The DNA pool is amplified by a biotinylated forward primer containing deoxyuridine and a phosphorylated reverse primer. The synthesized double-stranded amplicons are digested with Lambda exonuclease to selectively digest the non-biotinylated strand.

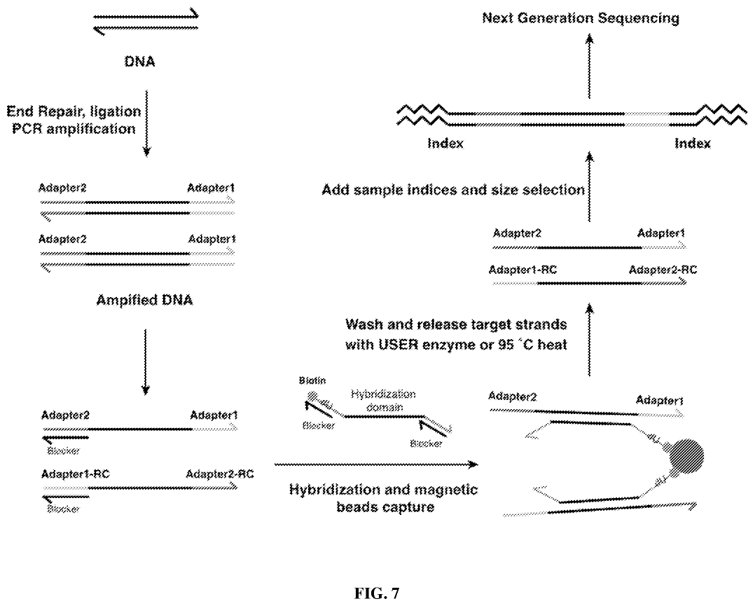

An exemplary workflow of SNP profiling by specialized hybrid-capture probe panel is shown in FIG. 7. The input DNA was end-repaired, followed by ligation reaction to add the universal adaptor sequences, according to the protocol described in NEBNext® Ultra™ II DNA Library Prep kit. The DNA was amplified using universal adaptors. If cfDNA is mixed with long DNA fragments, such as genomic DNA, DNA with length <500 bp is enriched by PCR with extension time between 1 second to 15 seconds and size selection as described herein. The amplified double-stranded DNA molecules are mixed with the biotinylated specialized hybrid-capture probes for SNP targeting, and blockers for universal regions. The mixture was incubated at 95° C. for 10 mins to denature double-strand DNA, followed by (65° C. 1 hr→47° C. 1 hr)×7, and 47° C. for 2 hr for hybridization. Streptavidin-coated magnetic beads are added to the mixture and incubated at 65° C. for 45 mins. After bead washing to remove unbound DNA, the bound DNA molecules are released by USER enzyme treatment or 95° C. heat. The bead washing and bound DNA elution can be performed using customized saline solution, or commercially available kits such as xGen® Lockdown® Reagents (Integrated DNA Technologies). Sample indices are added to the released DNA via PCR, and the products are sequenced by NGS.

### Example 8—Detection of Spike-in DNA

As a proof of concept for the detection of donor-derived cfDNA for organ transplant rejection, SNP profiling via a specialized hybrid-capture probe panel was carried out for a DNA sample with spike-in foreign DNA. Fragmented NA18537 genomic DNA (0.1 ng) is mixed with fragmented NA18562 genomic DNA (1 ng) in a 1:10 ratio. The SNP profiling was carried out as described in the previous section.

The spike-in DNA ratio is accurately detected via the SNP profiling. As summarized in Table 2, the fraction of molecules from NA18537 is 10.0% as calculated from the selected 53 SNP sites with different genotypes for NA18537 and NA18562. The observed spike-in fraction is close to the expected value (9.1%).

### Example 9—Quantification of Donor-Derived cfDNA Fraction

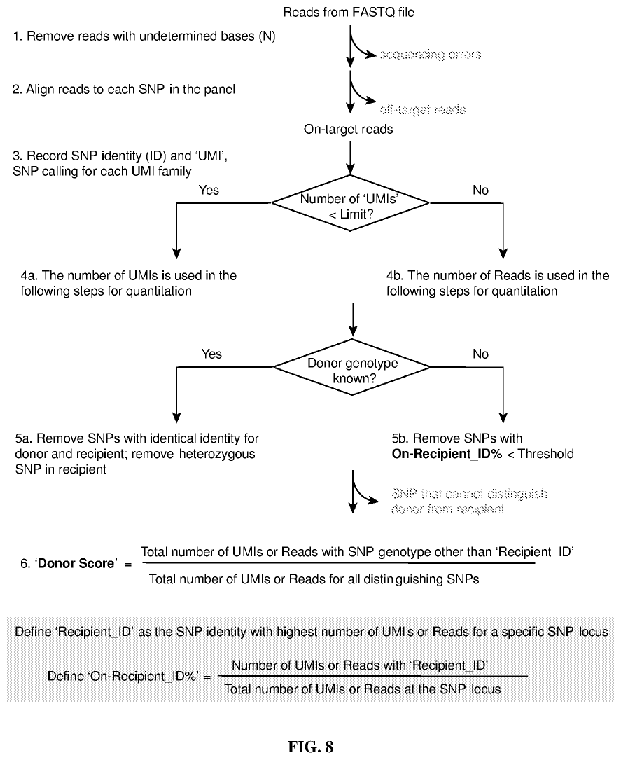

The workflow is summarized to quantitate the donor-derived DNA fraction in the DNA sample of organ recipient from SNP profiling NGS results (FIG. 8). The method can apply whether the donor genetic information is known or not.

The NGS reads without undetermined bases are firstly aligned to the reference genome for each probe in the SNP panel. The SNP genotypes and the UMIs are recorded. SNP genotype is called for each UMI family based on majority vote. If the number of UMIs is smaller than a threshold, which is set based on the input DNA amount, the UMI will be considered for data processing. However, if the number of UMIs is larger than the threshold, the number of fragmentation sites may not be sufficient to label each original molecule uniquely, and thus the UMI will not be considered for subsequent steps; NGS reads number will be used instead.

Distinguishing SNPs are selected. If the donor genotype is known, the SNPs with identical genotype between the donor and recipient will be discarded. Heterozygous SNPs in the recipient are also discarded. The remaining SNPs are considered as distinguishing SNPs. If the donor genotype is unknown, all the SNPs with an ‘On-Recipient_ID %’ larger than a threshold but no more than another threshold will be used as distinguishing SNPs. The thresholds are set between 80% and 99.99%. A donor Score for all distinguishing SNPs will be calculated to assess the donor-derived cfDNA fraction.

‘Recipient_ID’ is defined as the primary SNP genotype with the highest number of UMIs or Reads for a specific SNP locus.

‘On-Recipient_ID %’ is defined as:

$$On Recipient\_ID \% = \frac{Number of UMIs or Reads with ‘ Recipient\_ID ’}{Total number of UMIs or Reads at the SNP locus}$$

‘Donor Score’ for all distinguishing SNPs is defined as:

$$Donor Score = \frac{\begin{matrix}Total number of UMIs or Reads \\ with SNP genotype other than ‘ Recipient\_ID ’\end{matrix}}{Total number of UMIs or Reads for all distinguishing SNPs}$$

### Example 10—Quantification of Donor-Derived DNA Fraction with Low Input

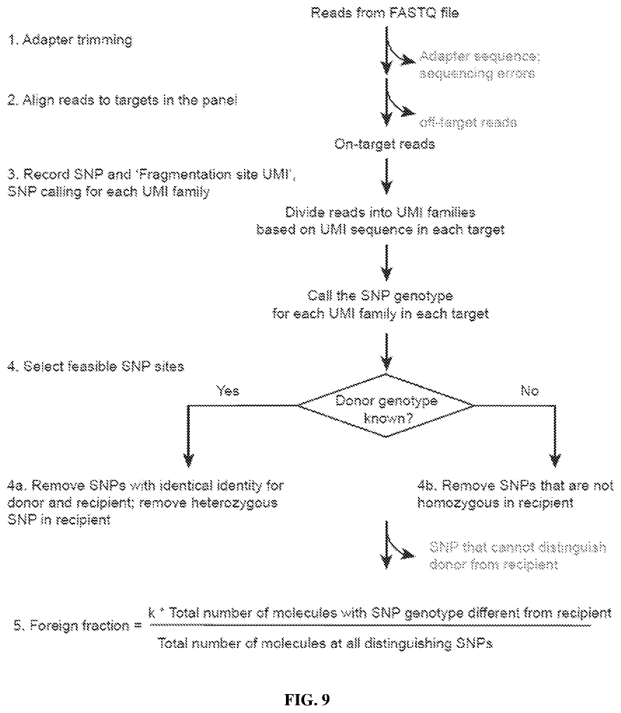

Another workflow is summarized to quantitate the foreign DNA fraction from low input (FIG. 9). The method can apply to the situations with known or unknown donor genetic information. The NGS reads without undetermined bases are first aligned to the reference genome for loci in the SNP panel. The SNP genotypes and the UMIs are recorded. At each SNP locus, the reads sharing the same UMI are presumed to originate from the same molecule and thus grouped together. The genotype is called for each UMI family at each SNP locus by majority vote: the genotype supported by more than 70% of reads is determined to be the genotype for the original molecule.

Distinguishing SNPs are selected. If the genotypes for both donor and recipient are known, the SNPs with identical identity between the donor and recipient will be discarded. Heterozygous SNPs in recipient are also discarded. The remaining SNPs for the foreign molecule fraction calculation are homozygous but different in donor and recipient. If the donor genotype is unknown, all the homozygous SNPs in the recipient will be considered for further calculation. The homozygous SNPs in the recipient can be determined using a gDNA sample obtained from buffy coat or buccal swab.

The total number of molecules with SNP genotype different from recipient is divided by the total number of molecules at all feasible SNPs. Because all recipient homozygous SNP loci are considered in case the donor genotype is unknown, there are three possible genotypes for donor: homozygous and same as recipient, homozygous but different from recipient, and heterozygous. A normalization factor k is required to calculate the foreign fraction to account for this. Since the population VAF is around 0.5 for all the SNPs, k=2 is used when donor genotype is known assuming donor and recipient are not related at all. When both donor and recipient genotypes are available, k=1 because only homozygous different cases are involved.

### Example 11—Quantification Validation with Serially Diluted Spike-in Samples

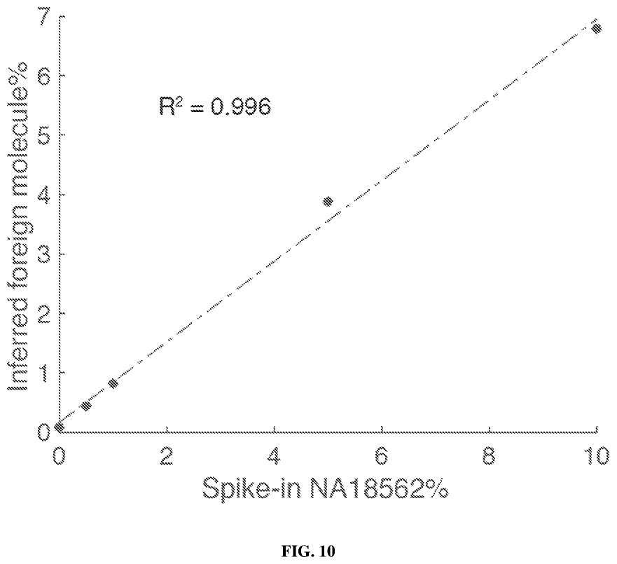

To evaluate quantitation performance, SNP profiling via the specialized hybrid-capture probe panel was carried out for a DNA sample with spike-in foreign DNA. Sheared NA18562 genomic DNA was mixed with sheared NA18537 genomic DNA in a 1:9 ratio to make a 10% spike-in The spike-in sample was serially diluted with NA18537 to make 5%, 1%, and 0.5% spike-in. Pure sheared NA18537 (0% spike-in) was also tested. The SNP profiling was carried out as described in the previous section, and quantitation was only based on the genotype of NA18537 without the genotype of “foreign molecule” as prior knowledge. Good linearity (R²=0.996) was shown in the plot of inferred foreign molecule percentage against the real spike-in value (FIG. 10), confirming the feasibility of calculating foreign molecule level without knowing the donor genotype. The inferred value was systematically lower than the spike-in value, indicating that the normalization factor k (k=2 here) might need to be adjusted because the assumption that donor and recipient are completely unrelated is not always true. Methods to determine the relatedness between the donor and recipient based on the recipient cfDNA sequencing data have been reported, and a similar approach may be used to adjust the normalization factor k for better quantitation. Even without such an adjustment, good linearity indicates that the occurrence of rejection can be monitored by comparing fold-increase to baseline.

### Example 12—Data on Healthy People and Non-Rejection Patients

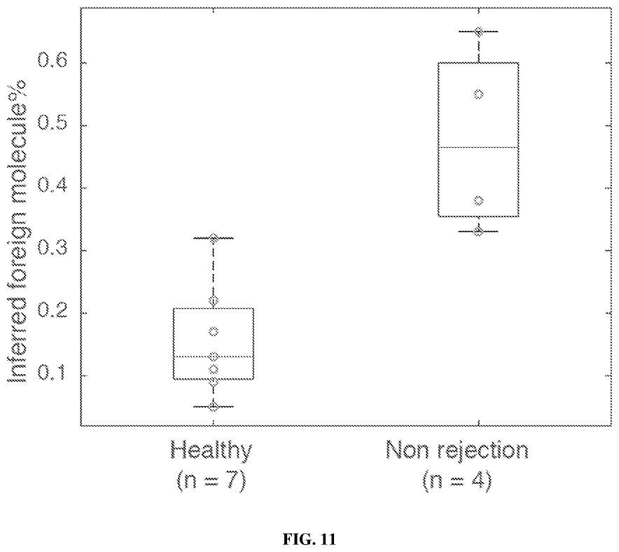

The foreign DNA quantitation method was tested using the fingerstick capillary blood samples from 7 healthy people without organ transplant and 4 organ transplant patients who showed no signs of rejection. The genotyping for recipients were determined using sheared genomic DNA. Paired venous blood was centrifuge, and the plasma layer was removed. Genomic DNA was extracted using the left mixture of buffy coat and red blood cell. It is note-worthy that though venous blood was collected here for genotyping, a less-invasive DNA source such as buccal swab can be used. In addition, the genotyping is only needed once so that venous blood collection in typical cfDNA extraction process can be avoided in the following monitoring tests. The inferred foreign molecule percentage summarized in a boxplot (FIG. 11) showed the baseline level of inferred foreign molecule in healthy people and the increased foreign molecule percentage in the 4 non-rejection organ transplant recipients (two kidney transplants and two lung transplants).

TABLE 1
NGS results for selective amplification of fragmented DNA
from mixture containing genomic DNA

| | Original Input: 1 ng | | Original Input: 0.1 ng | |
|---|---|---|---|---|
| | fragmented NA18537 + | | fragmented NA18537 + | |
| | 10 μg gDNA NA18562 | | 1 μg gDNA NA18562 | |
| | Molecules | Molecules | Molecules | Molecules |
| | from | from | from | from |
| SNP | NA18537 | NA18562 | NA18537 | NA18562 |
| rs1466275 | 2 | 48 | 0 | 27 |
| rs3827075 | 5 | 13 | 1 | 12 |
| rs62477615 | 3 | 7 | 5 | 6 |
| rs72648841 | 0 | 10 | 0 | 7 |
| rs1973028 | 2 | 11 | 0 | 12 |
| rs13040920 | 1 | 9 | 3 | 13 |
| rs4808092 | 1 | 25 | 4 | 18 |
| rs12514784 | 1 | 13 | 0 | 12 |
| rs12051158 | 2 | 10 | 0 | 5 |
| rs11488755 | 4 | 13 | 2 | 9 |
| rs8141354 | 2 | 9 | 0 | 8 |
| rs12051384 | 1 | 8 | 0 | 4 |
| rs4337500 | 0 | 12 | 2 | 10 |
| rs7449443 | 0 | 14 | 0 | 6 |
| rs7234216 | 1 | 9 | 0 | 10 |
| rs3753579 | 3 | 21 | 1 | 15 |
| rs34112762 | 0 | 16 | 2 | 9 |
| rs28620406 | 3 | 17 | 1 | 7 |
| rs2938220 | 3 | 6 | 0 | 7 |
| rs12454073 | 0 | 15 | 0 | 7 |
| rs7966098 | 4 | 31 | 0 | 19 |
| rs7184594 | 2 | 7 | 0 | 7 |
| rs2784915 | 1 | 1 | 0 | 4 |
| rs12914414 | 1 | 11 | 0 | 8 |
| rs2284051 | 0 | 8 | 0 | 6 |
| rs12112961 | 3 | 3 | 0 | 4 |
| rs8179470 | 3 | 17 | 3 | 5 |
| rs11117098 | 3 | 4 | 0 | 7 |
| rs12977522 | 0 | 11 | 2 | 8 |
| rs4993882 | 3 | 11 | 1 | 4 |
| rs8081225 | 3 | 19 | 0 | 14 |
| rs78826982 | 8 | 11 | 3 | 6 |
| rs6713590 | 2 | 8 | 0 | 3 |
| rs2279539 | 2 | 6 | 1 | 17 |
| rs28580460 | 0 | 7 | 0 | 5 |
| rs4497900 | 2 | 15 | 2 | 14 |
| rs2177320 | 2 | 15 | 2 | 4 |
| rs2363893 | 0 | 16 | 2 | 11 |
| rs60002603 | 1 | 6 | 0 | 5 |
| rs12548119 | 1 | 3 | 4 | 7 |
| rs1634454 | 1 | 5 | 2 | 7 |
| rs983740 | 0 | 2 | 1 | 4 |
| rs11081208 | 0 | 10 | 1 | 5 |
| rs506302 | 2 | 12 | 0 | 8 |
| rs905752 | 1 | 5 | 0 | 4 |
| rs701102 | 1 | 5 | 0 | 4 |
| rs11699942 | 1 | 10 | 0 | 3 |
| rs11134899 | 0 | 1 | 1 | 2 |
| rs338398 | 1 | 9 | 0 | 7 |
| rs934177 | 0 | 5 | 0 | 9 |
| rs12550370 | 1 | 8 | 1 | 8 |
| rs2114841 | 0 | 7 | 1 | 4 |
| rs10873477 | 4 | 13 | 2 | 2 |
| Sum | 87 | 588 | 50 | 429 |

| | | |
|---|---|---|
| Fraction of | 12.9% | 10.4% |
| molecules | | |
| from | | |
| NA18537 | | |
| Enrichment | 1289 | 1044 |
| fold | | |

TABLE 2
NGS results for detection of spike-in DNA by targeted
SNP panel

| | Original Input: 0.1 ng fragmented | |
|---|---|---|
| | NA18537 + 1 ng fragmented | |
| | NA18562 | |
| | Molecules from | Molecules from |
| SNP | NA18537 | NA18562 |
| rs1466275 | 1 | 19 |
| rs3827075 | 1 | 16 |
| rs62477615 | 9 | 11 |
| rs72648841 | 1 | 18 |
| rs1973028 | 1 | 17 |
| rs13040920 | 1 | 10 |
| rs4808092 | 2 | 17 |
| rs12514784 | 2 | 17 |
| rs12051158 | 2 | 14 |
| rs11488755 | 2 | 4 |
| rs8141354 | 2 | 18 |
| rs12051384 | 2 | 13 |
| rs4337500 | 2 | 8 |
| rs7449443 | 0 | 10 |
| rs7234216 | 1 | 3 |
| rs3753579 | 0 | 20 |
| rs34112762 | 1 | 8 |
| rs28620406 | 1 | 7 |
| rs2938220 | 0 | 11 |
| rs12454073 | 0 | 12 |
| rs7966098 | 1 | 14 |
| rs7184594 | 2 | 4 |
| rs2784915 | 0 | 7 |
| rs12914414 | 0 | 3 |
| rs2284051 | 0 | 5 |
| rs12112961 | 1 | 14 |
| rs8179470 | 1 | 7 |
| rs11117098 | 0 | 17 |
| rs12977522 | 0 | 1 |
| rs4993882 | 1 | 6 |
| rs8081225 | 2 | 15 |
| rs78826982 | 2 | 6 |
| rs6713590 | 0 | 2 |
| rs2279539 | 0 | 5 |
| rs28580460 | 1 | 10 |
| rs4497900 | 4 | 6 |
| rs2177320 | 1 | 9 |
| rs2363893 | 1 | 9 |
| rs60002603 | 0 | 11 |
| rs12548119 | 0 | 4 |
| rs1634454 | 0 | 6 |
| rs983740 | 0 | 4 |
| rs11081208 | 0 | 5 |
| rs506302 | 1 | 10 |
| rs905752 | 0 | 10 |
| rs701102 | 0 | 6 |
| rs11699942 | 1 | 10 |
| rs11134899 | 1 | 2 |
| rs338398 | 1 | 9 |
| rs934177 | 1 | 18 |
| rs12550370 | 0 | 5 |
| rs2114841 | 0 | 2 |
| rs10873477 | 2 | 2 |
| Sum | 55 | 497 |

| | |
|---|---|
| Fraction of molecules | 10.0% |
| from NA18537 | |
| Expected fraction | 9.1% |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. Appln. Publn. No. 2016/0145682
Park et al., “Integrated Kidney Exosome Analysis for the Detection of Kidney Transplant Rejection,” ACS Nano., 11:11041-11046, 2017.
Suthanthiran et al., “Urinary-Cell mRNA Profile and Acute Cellular Rejection in Kidney Allografts,” N. Engl. J. Med., 369:29-31, 2013.
Vallabhajosyula et al., “Tissue-specific exosome biomarkers for noninvasively monitoring immunologic rejection of transplanted tissue,” J. Clin. Invest., 127:1375-1391, 2017.

What is claimed is:

1. A method of selectively amplifying short DNA fragments in a DNA sample that comprises both long and short DNA fragments, the method comprising:
(a) ligating a universal adaptor oligonucleotide to each end of the long and short DNA fragments, thereby generating adaptor-modified long and short DNA fragments,
(b) selectively amplifying the adaptor-modified short DNA fragments by performing PCR with an extension time of between about 1 second and about 15 seconds and using oligonucleotide primers that hybridize to the universal adaptor, thereby generating amplified short DNA fragments, and
(c) performing size selection to isolate the amplified short DNA fragments.

2. The method of claim 1, wherein the short DNA fragments have a length between about 50 nucleotides and 400 nucleotides.

3. The method of claim 1-2, wherein the PCR in step (b) is performed with an annealing time of between about 1 second and about 30 seconds.

4. The method of any one of claims 1-3, wherein the DNA sample comprises cell-free DNA (cfDNA).

5. The method of claim 4, wherein the short DNA fragments comprise cell-free DNA (cfDNA).

6. The method of any one of claims 1-5, wherein the DNA sample comprises DNA extracted from total blood.

7. The method of any one of claims 1-5, wherein the DNA sample is extracted from a buccal swab or urine.

8. The method of any one of claims 1-7, wherein, prior to step (a), the long and short DNA fragments are subjected to end-repair.

9. The method of any one of claims 1-8, wherein, prior to step (b), the adaptor-modified long and short DNA fragments are column purified.

10. The method of any one of claims 1-9, wherein the universal adaptors comprise, from 5′ to 3′, a region that is complementary to the oligonucleotide primers and a region that is not complementary to the oligonucleotide primers.

11. The method of any one of claims 1-10, wherein the size selection of step (c) comprises gel electrophoresis purification or beads-based purification.

12. The method of any one of claims 1-11, further comprising (d) sequencing the amplified short DNA fragments.

13. The method of claim 12, wherein the sequencing in step (d) is next-generation sequencing.

14. The method of claim 13, wherein the next-generation sequencing is paired-end sequencing or single-read sequencing.

15. The method of claim 14, further comprising (e) enriching the amplified short DNA fragment sequences by (1) aligning the sequences to a reference genome to determine the amplicon length and (2) removing any sequences with an amplicon length greater than 400 nucleotides.

16. A method of analyzing single nucleotide polymorphisms (SNPs) in a DNA sample, the method comprising
(a) hybridizing the DNA sample to a mixture of hybrid-capture probes, wherein at least 80% of the hybrid-capture probes correspond, independently, to a genomic region having a SNP with a population minor allele frequency of greater than 25%, wherein each genomic region:
(1) occurs no more than 10 times in the genome;
(2) has a GC content of between about 0.25 and about 0.75; and
(3) does not contain any string of a single base that is longer than 4 nucleotides,
thereby generating capture probe-bound DNA;
(b) isolating the hybrid-capture probe-bound DNA;
(c) ligating a universal adaptor oligonucleotide to each end of the hybrid-capture probe-bound DNA;
(d) amplifying the hybrid-capture probe-bound DNA using primers that hybridize to the adaptor sequences, thereby generating amplified DNA; and
(e) sequencing the amplified DNA.

17. The method of claim 16, wherein each genomic region comprises the 80 nucleotides surrounding the SNP.

18. The method of claim 17, wherein each genomic region is unique in the genome.

19. The method of any one of claims 16-18, wherein the method analyzes between about 500 and about 1,000,000 SNPs.

20. The method of any one of claims 16-19, wherein the DNA sample is amplified prior to step (a), thereby generating an amplified double-stranded DNA sample.

21. The method of claim 20, wherein the DNA sample is amplified according to the method of any one of claims 1-15.

22. The method of claim 20 or 21, wherein the amplified DNA sample comprises DNA fragments having a length of between about 50 nucleotides and about 400 nucleotides.

23. The method of claim 20, wherein the amplified double-stranded DNA sample is denatured prior to step (a), thereby generating an amplified single-stranded DNA sample.

24. The method of claim 23, wherein the amplified double-stranded DNA sample is denatured by heating the amplified double-stranded DNA sample at a temperature of at least 80° C. for at least 2 minutes.

25. The method of claim 23, wherein the amplified double-stranded DNA sample is denatured by chemical denaturation.

26. The method of claim 25, wherein the chemical denaturation comprises incubating the amplified double-stranded DNA sample with sodium hydroxide.

27. The method of claim 23, wherein the amplified double-stranded DNA sample is denatured by enzymatic denaturation.

28. The method of any one of claims 16-27, wherein the sequencing in step (d) is next-generation sequencing.

29. The method of claim 28, wherein the next-generation sequencing is paired-end sequencing.

30. The method of claim 28, wherein the next-generation sequencing is single-read sequencing.

31. The method of any one of claims 16-30, wherein the isolating in step (b) comprises solid-phase capture of the hybrid-capture probe-bound DNA.

32. The method of claim 31, wherein the solid-phase capture of the hybrid-capture probe-bound DNA comprises incubating the hybrid-capture probe-bound DNA with streptavidin-coated beads.

33. The method of claim 31, wherein the isolating in step (b) further comprises separating, washing, and releasing the hybrid-capture probe-bound DNA.

34. The method of claim 33, wherein separating comprises magnetic separation or centrifugation.

35. The method of claim 33, wherein releasing comprises heating the captured hybrid-capture probe-bound DNA at at least 80° C. for at least 2 minutes.

36. The method of claim 33, wherein the hybrid-capture probes further comprise an enzyme recognition moiety.

37. The method of claim 36, wherein the enzyme recognition moiety is a deoxyuridine.

38. The method of claim 36, wherein releasing comprises performing enzymatic cleavage of the enzyme recognition moiety.

39. The method of claim 37, wherein releasing comprises incubating the captured hybrid-capture probe-bound DNA with a USER enzyme.

40. The method of any one of claims 16-39, wherein the DNA sample comprises cell-free DNA (cfDNA).

41. The method of claim 40, wherein the cfDNA is amplified prior to step (a).

42. The method of any one of claims 16-41, wherein the hybrid-capture probes are biotinylated.

43. The method of any one of claims 16-41, wherein the hybrid-capture probes are hybridized to a biotinylated oligonucleotide.

44. A method of determining the number of unique cfDNA fragments in a sample containing less than 4 ng of cfDNA and/or correcting errors from amplification and sequencing, the method comprising:
(a) amplifying the cfDNA fragments;
(b) sequencing the amplified cfDNA fragments using paired-end next-generation sequencing;
(c) aligning the sequences to a reference genome, and determining the start and end position of each sequenced cfDNA fragment;
(d) separating the sequences by the genomic loci they are aligned to, and calling the fragment sequence based on majority vote of all the sequencing reads with the same start and end positions; and
(e) counting the number of unique start and end positions from among the sequenced cfDNA fragments, thereby determining the number of cfDNA fragments at each genomic locus of interest corresponding to each different genotype in the sample.

45. The method of claim 44, wherein the start and end positions are determined by next-generation sequencing paired-end reads.

46. A method of determining the number of unique cfDNA fragments in a sample containing more than 4 ng of cfDNA and/or correcting errors from amplification and sequencing, the method comprising:
(a) ligating an adaptor nucleic acid to each end of each cfDNA fragment, wherein the adaptor nucleic acid comprises a degenerate sequence;
(b) amplifying the adaptor-ligated cfDNA fragments;
(c) sequencing the amplified cfDNA fragments using paired-end next-generation sequencing;
(d) aligning the sequences to a reference genome, and determining the start and end position of each sequenced cfDNA fragment;
(e) separating the sequences by the genomic loci they are aligned to, and calling the fragment sequence based on majority vote of all the sequencing reads with the same combined start and end positions and degenerate sequences; and
(f) counting the number of unique combined start and end positions and degenerate sequences from among the sequenced cfDNA fragments, thereby determining the number of cfDNA fragments at each genomic locus of interest corresponding to each different genotype in the sample.

47. The method of claim 46, wherein the start and end positions are determined by next-generation sequencing paired-end reads.

48. A method of monitoring organ transplant rejection by SNP profiling, the method comprising:
(a) extracting cell-free DNA (cfDNA) and genomic DNA (gDNA) from a DNA sample obtained from an organ transplant recipient;
(b) selectively amplifying short fragments of cell-free DNA using the method of any one of claims 1-15;
(c) obtaining sequence reads for at least 500 single nucleotide polymorphisms (SNPs) in the amplified cell-free DNA using the method of any one of claims 16-43; and
(d) quantifying a fraction of the organ transplant donor-derived cell-free DNA versus the DNA of the organ recipient.

49. The method of claim 48, wherein the cfDNA and gDNA are extracted from whole blood.

50. The method of claim 49, wherein the cfDNA and gDNA are extracted from a low-volume of whole blood.

51. The method of claim 49, wherein the extraction in step (a) further comprises plasma separation.

52. The method of claim 49, wherein the whole blood is venous blood.

53. The method of claim 49, wherein the whole blood is obtained from a finger-stick.

54. The method of claim 48, wherein the cfDNA and gDNA are extracted from a buccal swab.

55. The method of any one of claims 48-54, wherein step (c) comprises analyzing between 500 and about 1,000,000 SNPs.

56. The method of any one of claims 48-55, wherein step (d) comprises:
(1) removing sequencing reads that comprise undetermined bases; and
(2) determining the number of unique DNA fragments for each SNP locus and each genotype.

57. The method of claim 56, wherein determining the number of unique DNA fragments for each SNP locus and each genotype comprises performing the method of any one of claims 44-47.

58. The method of any one of claims 48-57, wherein the at least 500 SNPs consists of SNPs for which the organ transplant recipient is homozygous.

59. The method of any one of claims 48-58, wherein the at least 500 SNPs consists of SNPs for which the organ transplant recipient and the organ donor are not identical.

60. The method of any one of claims 48-59, wherein if the fraction of the short fragments of cell-free DNA that correspond to the genomic DNA of the organ transplant donor is above a normal range or increases over time, then the organ transplant recipient is considered to be rejecting the transplanted organ.
